(19) United States
(12) Patent Application Publication
(10) Pub. No.: US 2003/0113921 A1
(43) Pub. Date: Jun. 19, 2003

(54) Nucleic acid sequences capable of improving homologous recombination in plants and plant plastids

(76) Inventors: Larry A. Gilbertson, Chesterfield, MO (US); Jeffrey M. Staub, Wildwood, MO (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 10/199,621

(22) Filed: Jul. 19, 2002

Related U.S. Application Data
(60) Provisional application No. 60/306,680, filed on Jul. 20, 2001.

(51) Int. Cl. C12N 15/74; C07H 21/04

(52) U.S. Cl. 435/475; 435/320.1; 536/23.1

(57) ABSTRACT

A method for improved plastid transformation efficiency via homologous recombination and nucleic acid sequences useful therefore is provided. Nucleic acid sequences comprising a 5 base pair recombination sequence motif or multiple direct repeats thereof that increase the frequency of integration of a selected transgene through plastid transformation by homologous recombination are provided.

### FIELD OF THE INVENTION

This invention relates in general to plant and plant plastid transformation and more particularly to nucleic acid sequences useful in improving homologous recombination in such plant or plant plastid and methods of using such nucleic acid sequences to transform plants and plant plastids.

### BACKGROUND OF THE INVENTION

Homologous recombination is believed to be the standard mechanism by which foreign genes are inserted into a plastid genome (Maliga, 1993; Maliga et al., 1994). Transgenes are typically introduced into leaf cell chloroplasts by particle bombardment, where integration of the foreign DNA is directed by homologous recombination to a predetermined location in the genome. Plastids have a polyploid genetic system; with up to 100 plastids per cell carrying up to 100 plastid genomes each, for a total of 10,000 plastid DNA (ptDNA) molecules in a leaf cell (Bendich, 1987). Stable transformation is achieved through a process of selection, amplification and subsequent segregation and sorting of a selectable marker until homoplasmy is achieved (Maliga, 1993). Not only has homologous recombination in plastids been exploited for the study of gene function through gene insertion, disruption and deletion (reviewed in (Bogorad, 2000) but also for marker rescue (Staub and Maliga, 1995) and antibiotic marker gene excision (Fischer et al., 1996; Iamtham and Day, 2000).

A typical plastid transformation vector has the desired transgene (or gene of interest) flanked by fragments of plastid DNA that have homology to sequences in the plastid genome. The efficiency of plastid transformation events by this method is low and requires considerable effort to identify and obtain a plant having a desired transformed plant plastid (a transplastomic plant). Thus, it would be desirable to identify and implement methods and/or compositions capable of improving plastid transformation methodology in a manner that increases the frequency of integration of the desired transgene by homologous recombination.

### SUMMARY OF THE INVENTION

This invention relates to a method for improved plastid transformation efficiency via homologous recombination and nucleic acid sequences useful therefor. In one aspect of the invention, a nucleic acid sequence comprising a 5 base pair recombination sequence motif or multiple direct repeats thereof that increase the frequency of integration of a selected transgene through plastid transformation by homologous recombination is provided. The recombination sequence motif generally comprises the sequence 5′-TATTA-3′, its complement 3′-TAATA -5′ and imperfect repeats of the motif that are changed by one nucleotide (e.g. 5′-GATTA-3′) or a plurality of such motifs, more particularly (TATTA)ₙ or (TAATA)ₙ, where n is between about 2 and about 40 and wherein the recombination sequence motifs may be interspersed with other nucleotides such as in X-(TATTA)ₚ-X-(TATTA)ₚ-X, where X is any sequence of nucleotides between about 1 and about 10 nucleotides in length and p is between about 1 and about 20. In a further embodiment of the invention, the recombination sequence motif comprises at least one segment of AᵣTᵣ rich repeated sequences where r is between about 5 and about 50 and the AT rich segment is between about 20 and about 100 base pairs in length.

A still further aspect of the invention provides plastid transformation vectors comprising a transgene to be inserted into the plastid genome whereby the transgene is cloned adjacent to or directly within a recombination sequence motif of the present invention. The transformation vector may optionally contain additional flanking homologous sequences.

In a yet further aspect of the invention, a parental transplastomic plant line is provided that comprises an engineered recombination sequence motif of the present invention within its plastid genome that is used as an integration site for further transformations.

Among the many aims and objectives of the present invention include the provision of a method of plastid transformation providing for increased frequency of integration of a selected transgene by homologous recombination and nucleic acid sequences and vectors useful therefor. Transplastomic plants prepared by the method of the present invention are also provided. Other and further aims and objects of the invention will become apparent from the drawing figures, descriptions and claims that follow.

### BRIEF DESCRIPTION OF THE DRAWING FIGURES

FIG. 1 shows a schematic version of a vector construct containing the recombination sequence motif of the present invention adjacent to a selected transgene (GOI);

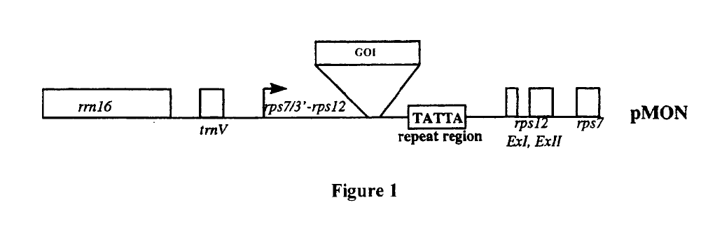

FIG. 2 shows a schematic version of a vector construct containing a selected transgene (GOI) within the recombination sequence motif of the present invention and the recombination with a recipient plastid genome (ptDNA);

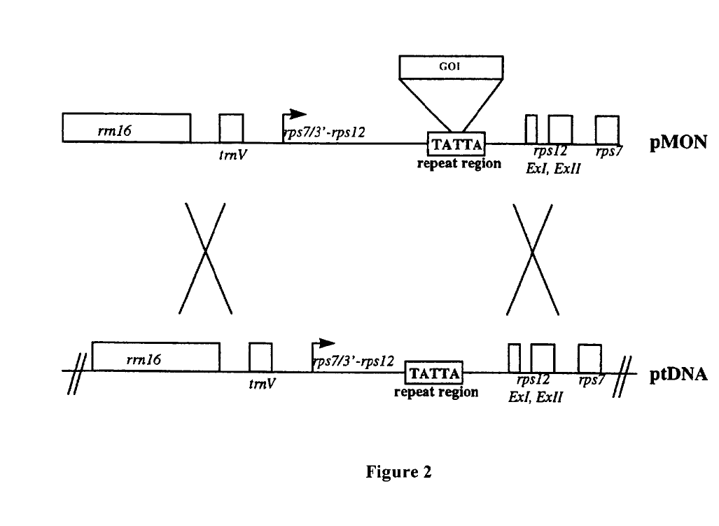

FIG. 3 shows a schematic version of a vector construct containing a plurality of the recombination sequence motif flanking the selected transgene (GOI) and cloned into the homolgous flanking region as well as homologous flanking sequence and the recombination locations with the recipient plastid DNA (ptDNA);

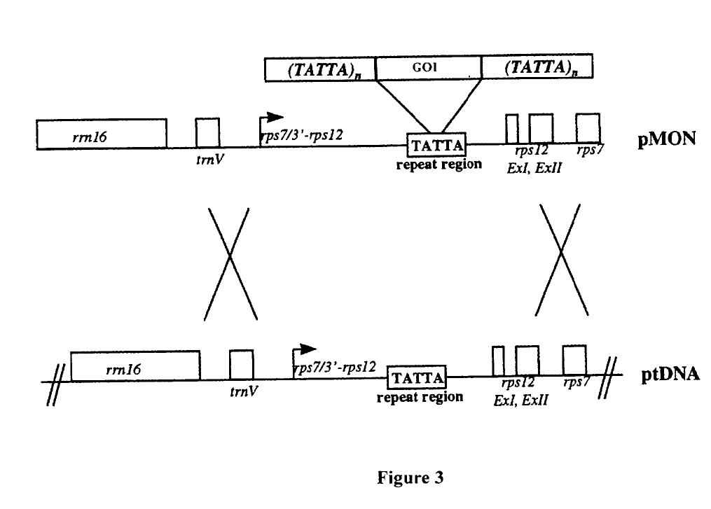

FIG. 4 shows a schematic version of a vector construct containing only a plurality of engineered recombination sequence motifs flanking the selected transgene (GOI) and not cloned into a homologous flanking region and a mechanism of cloning directly into the repeat region in the plastid DNA;

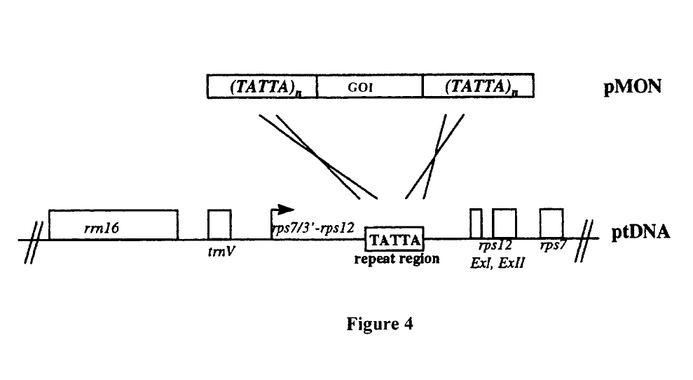

FIG. 5 is a schematic of integration of a fragment of DNA into a plastid genome by homologous recombination using a fragment engineered to contain TATTA repeats such that the repeats are integrated along with one or more transgenes;

FIG. 6 shows a schematic version of a vector construct pMON49219 containing maize genes rbcL, psai and ORF185 Large Single Copy flanking region clone, including the unique NotI site used for transgene insertion;

FIG. 7 shows a schematic version of a vector construct pMON38722 containing maize genes rrn16 (16S rDNA), trnV (tRNA-Val), ORF85 ORF58, and rps12 Exons I and II in the Inverted Repeat flanking region clone, including unique NotI site used for transgene insertion;

FIG. 8 provides a schematic representation of plasmid pMON53119, the aadA selectable marker is flanked by lox sites in direct repeat orientation. The aadA gene is cloned between the GFP gene and its promoter (Prrn), preventing GFP expression. Note opposite orientation of aadA relative to the GFP transgene to prevent readthrough transcription into GFP. The chimeric genes are inserted between the plastid rps7/3′-rps12 operon (rps) and the trnV^{(GAC)} and rrn16 genes used as homologous flanking regions for targeting into the Inverted Repeat region of the tobacco plastid genome. The BglII (Bg) and EcoRI (RI) restriction sites denote the endpoints of the plastid DNA region in pMON53119;

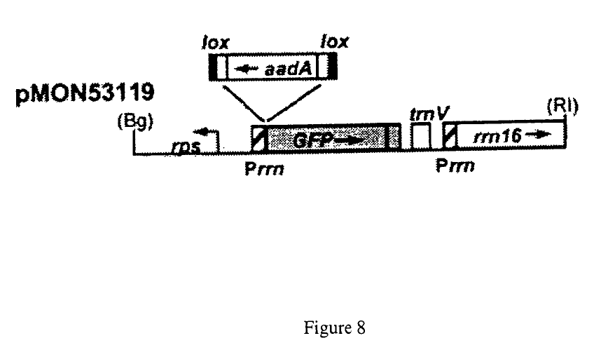

FIG. 9 illustrates the sequence of the recombinational hotspot region described herein. Note the presence of multiple copies of the directly repeated sequence motif, TATTA (underlined) in the plastid genome. The wild-type loxP recognition sequence is shown underneath with the Cre cleavage sites marked. The junctional nucleotides of the recombination (arrows) between the sequence in the hotspot region (asterisk) and the corresponding loxP sequence in lines Nt-Act2-53119-38, Nt-e35S-53119-10 and Nt-Act2-53119-40 are also shown.

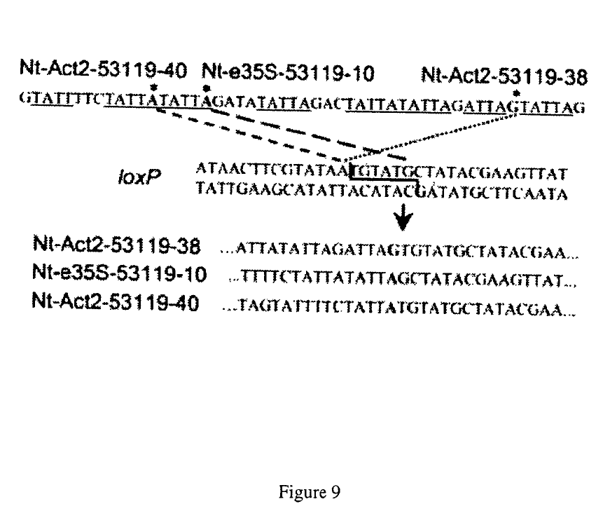

### DETAILED DESCRIPTION OF THE INVENTION

In accordance with the subject invention, constructs and methods are provided for obtaining plants having transformed plastids (transplastomic plants). The methods and constructs of the present invention provide a novel means for increasing the frequency of integration of a selected nucleic acid sequence into a predetermined site in a plastid genome. A novel recombination sequence motif has been discovered that permits homologous recombination to occur more frequently when included in the homologous flanking regions of a plastid transformation vector or fully comprises such homologous flanking regions by means of a plurality of the recombination sequence motifs.

It is known that a region of the plastid genome contains numerous direct repeats of the recombination sequence motif 5′-TATTA-3′ or a variation thereof involving an AT rich sequence motif. The genomic location of the TATTA repeat region is downstream of the transcriptional start site of the rps7/3′-rps 12 operon promoter in tobacco chloroplasts (positions 101756-101820 or 140821-140875 in the Inverted Repeat of the tobacco chloroplast genome, Genbank accession Z00044). It has been found that recombination occurred frequently between these TATTA direct repeats and a wild-type loxP site in transplastomic plants, when the Cre recombinase was expressed from a nuclear-encoded plastid targeted construct.

The plastid genomic sequence that carries the TATTA direct repeats resides in the Inverted Repeat region of the plastid genome, and so is present in two copies per plastid genome. No other similar repeated sequences are known in the tobacco plastid genome. In an analogous region of the maize plastid genome, however, a region carrying directly repeated TAATA sequences (complementary to TATTA and thus considered to be identical but on opposite strands of the DNA) was identified and it is likely that such a recombination sequence motif will be found by inspection of other plastid genomes from other plants given the evolutionary relationship among plastid genomes using known methods in the art. Thus, long regions (greater than 20 bp) of AT-rich repeated sequences may serve as recombinational enhancers in plastids and would be useful to improve the efficiency of plastid transformation. It is thus to be expected that the presence of the recombination sequence motif of the present invention either in combination with other known plastid flanking regions capable of initiating homologous recombination or alone or as a plurality of such recombination sequence motifs may increase plastid transformation efficiency of integration of the selected nucleic acid (the gene of interest).

The following definitions and methods are provided to better define, and to guide those of ordinary skill in the art in the practice of, the present invention. Unless otherwise noted, terms are to be understood according to conventional usage by those of ordinary skill in the relevant art. The nomenclature for DNA bases as set forth at 37 CFR §1.822 is used. The standard one- and three-letter nomenclature for amino acid residues is used.

A first nucleic acid sequence is “operably linked” with a second nucleic acid sequence when the sequences are so arranged that the first nucleic acid sequence affects the function of the second nucleic-acid sequence. Preferably, the two sequences are part of a single contiguous nucleic acid molecule and more preferably are adjacent. For example, a promoter is operably linked to a gene if the promoter regulates or mediates transcription of the gene in a cell.

Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers.

A “synthetic nucleic acid sequence” can be designed and chemically synthesized for enhanced expression in particular host cells and for the purposes of cloning into appropriate vectors. Host cells often display a preferred pattern of codon usage (Murray et al., 1989). Synthetic DNAs designed to enhance expression in a particular host should therefore reflect the pattern of codon usage in the host cell. Computer programs are available for these purposes including but not limited to the “BestFit” or “Gap” programs of the Sequence Analysis Software Package, Genetics Computer Group, Inc., University of Wisconsin Biotechnology Center, Madison, Wis. 53711.

“Amplification” of nucleic acids or “nucleic acid reproduction ” refers to the production of additional copies of a nucleic acid sequence and is carried out using polymerase chain reaction (PCR) technologies. A variety of amplification methods are known in the art and are described, inter alia, in U.S. Pat. Nos. 4,683,195 and 4,683,202 and in PCR Protocols: A Guide to Methods and Applications, ed. Innis et al., Academic Press, San Diego, 1990. In PCR, a primer refers to a short oligonucleotide of defined sequence which is annealed to a DNA template to initiate the polymerase chain reaction.

“Transformed”, “transfected”, or “transgenic” refers to a cell, tissue, organ, or organism into which has been introduced a foreign nucleic acid or heterologous polynucleotide, such as a recombinant vector. Preferably, the introduced nucleic acid is stably integrated into the genomic DNA of the recipient cell, tissue, organ or organism such that the introduced nucleic acid is inherited by subsequent progeny. A “transgenic” or “transformed” cell or organism also includes progeny of the cell or organism and progeny produced from a breeding program employing such a “transgenic” plant as a parent in a cross and exhibiting an altered phenotype resulting from the presence of a recombinant construct or vector.

The term “gene” refers to chromosomal DNA, plasmid DNA, cDNA, synthetic DNA, or other DNA that encodes a peptide, polypeptide, protein, or RNA molecule, and regions flanking the coding sequence involved in the regulation of expression. Some genes can be transcribed into mRNA and translated into polypeptides (structural genes); other genes can be transcribed into RNA (e.g. rRNA, tRNA); and other types of gene function as regulators of expression (regulator genes).

“Expression” of a gene refers to the transcription of a gene to produce the corresponding mRNA and translation of this mRNA to produce the corresponding gene product, i.e., a peptide, polypeptide, or protein. Gene expression is controlled or modulated by regulatory elements including 5′ regulatory elements such as promoters.

“Genetic component” refers to any nucleic acid sequence or genetic element which may also be a component or part of an expression vector. Examples of genetic components include, but are not limited to promoter regions, 5′ untranslated leaders, introns, genes, 3′ untranslated regions, and other regulatory sequences or sequences which affect transcription or translation of one or more nucleic acid sequences.

The terms “recombinant DNA construct”, “recombinant vector”, “expression vector” or “expression cassette” refer to any agent such as a plasmid, cosmid, virus, BAC (bacterial artificial chromosome), autonomously replicating sequence, phage, or linear or circular single-stranded or double-stranded DNA or RNA nucleotide sequence, derived from any source, capable of genomic integration or autonomous replication, comprising a DNA molecule in which one or more DNA sequences and/or genetic components have been linked in a functionally operative manner using well-known recombinant DNA techniques.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species, or, if from the same species, is substantially modified from its original form by deliberate human intervention.

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention. A “recombinant” nucleic acid is made by an artificial combination of two otherwise separated segments of sequence, e.g., by chemical synthesis or by the manipulation of isolated segments of nucleic acids by genetic engineering techniques. Techniques for nucleic-acid manipulation are well-known (see for example Sambrook et al., Molecular Cloning: A Laboratory Manual, Cold Spring Harbor Press, 1989; Mailga et al., Methods in Plant Molecular Biology, Cold Spring Harbor Press, 1995; Birren et al., Genome Analysis: volume 1, Analyzing DNA, (1997), volume 2, Detecting Genes, (1998), volume 3, Cloning Systems, (1999) volume 4, Mapping Genomes, (1999), Cold Spring Harbor, N.Y.).

By “host cell” is meant a cell which contains a vector and supports the replication, and/or transcription or transcription and translation (expression) of the expression construct. Host cells for use in the present invention can be prokaryotic cells, such as E. coli, or eukaryotic cells such as yeast, plant, insect, amphibian, or mammalian cells. Preferably, host cells are monocotyledenous or dicotyledenous plant cells.

As used herein, the term “plant” includes reference to whole plants, plant organs (for example, leaves, stems, roots, etc.), seeds, and plant cells and progeny of same. Plant cell, as used herein includes, without limitation, seeds suspension cultures, embryos, meristematic regions, callus tissue, leaves roots shoots, gametophytes, sporophytes, pollen, and microspores. The class of plants which can be used in the methods of the present invention is generally as broad as the class of higher plants amenable to transformation techniques, including both monocotyledenous and dicotyledenous plants. Particularly preferred plants include tobacco, Arabidopsis, Brassica, soybean, rice, wheat, tomato, potato, sunflower, canola and corn.

As used herein, “transplastomic” refers to a plant cell having a heterologous nucleic acid introduced into the plant cell plastid. The introduced nucleic acid may be integrated into the plastid genome, or may be contained in an autonomously replicating plasmid. Preferably, the nucleic acid is integrated into the plant cell plastid's genome.

The term “Introduced” in the context of inserting a nucleic acid sequence into a cell, means “transfection”, or “transformation” or “transduction” and includes reference to the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (for example, transfected mRNA).

In developing the constructs of the invention, the various fragments comprising the regulatory regions and open reading frame may be subjected to different processing conditions, such as ligation, restriction enzyme digestion, PCR, in vitro mutagenesis, linkers and adapters addition, and the like. Thus, nucleotide transitions, transversions, insertions, deletions, or the like, may be performed on the DNA that is employed in the regulatory regions or the nucleic acid sequences of interest for expression in the plastids. Methods for restriction digests, Klenow blunt end treatments, ligations, and the like are well known to those in the art and are described, for example, by Maniatis et al. (in Molecular cloning: a laboratory manual (1982) Cold Spring Harbor Laboratory Press, Cold Spring Harbor, N.Y.).

During the preparation of the constructs, the various fragments of DNA will often be cloned in an appropriate cloning vector, which allows for amplification of the DNA, modification of the DNA or manipulation by joining or removing of sequences, linkers, or the like. Normally, the vectors will be capable of replication in at least a relatively high copy number in E. coli. A number of vectors are readily available for cloning, including such vectors as pBR322, pUC series, M13 series, and pBluescript (Strategene; La Jolla, Calif.).

The constructs for use in the methods of the present invention are prepared to direct the expression of the nucleic acid sequences directly from the host plant cell plastid. Examples of such constructs and methods are known in the art and are generally described, for example, in Svab et al. (1990) Proc. Natl. Acad. Sci. USA 87:8526-8530 and Svab and Maliga (1993) Proc. Natl. Acad. Sci. USA 90:913-917 and in U.S. Pat. No. 5,693,507.

The skilled artisan will recognize that any convenient element that is capable of initiating transcription in a plant cell plastid, also referred to as “plastid functional promoters,” can be employed in the constructs of the present invention. A number of plastid functional promoters are available in the art for use in the constructs and methods of the present invention. Such promoters include, but are not limited to, the promoter of the D1 thylakoid membrane protein, psbA (Staub et al. EMBO Journal, 12(2):601-606, 1993), and the 16S rRNA promoter region, Prrn (Svab and Maliga,1993, Proc. Natl. Acad. Sci. USA 90:913-917). The expression cassette(s) can include additional elements for expression of the protein, such as transcriptional and translational enhancers, ribosome binding sites, and the like.

As translation is a limiting step for plastid transgene expression, a variety of translational control elements need to be tested for efficacy. Efficient transgene translation will ensure that the markers used for selection of plastid transformed cells will function. Examples of such translational enhancing sequences include the heterologous bacteriophage T7 gene 10 leader (G10L) (Staub et al., 2000 Nature Biotech. 18:333-338); an additional translational fusion of 14 amino acids of the green fluorescent protein (14aaGFP) that has also been shown to enhance translation, may be used in addition to the G10L; in other cases, the “downstream box” sequence from the bacteriophage T7 gene 10 coding region (EC DB), which also enhances translation, may be used in addition to the G10L.

Regulatory transcript termination regions may be provided in the expression constructs of this invention as well. Transcript termination regions may be provided by any convenient transcription termination region derived from a gene source; e.g., the transcript termination region that is naturally associated with the transcript initiation region. The skilled artisan will recognize that any convenient transcript termination region that is capable of terminating transcription in a plant cell may be employed in the constructs of the present invention.

The expression cassettes for use in the methods of the present invention also preferably contain additional nucleic acid sequences providing for the integration into the host plant cell plastid genome or for autonomous replication of the construct in the host plant cell plastid. Preferably, the plastid expression constructs contain regions of homology for integration into the host plant cell plastid. The regions of homology employed can target the constructs for integration into any region of the plastid genome; preferably the regions of homology employed target the construct to either the inverted repeat region of the plastid genome or the large single copy region. Where more than one construct is to be used in the methods, the constructs can employ the use of the regions of homology to target the insertion of the construct into the same or a different position of the plastid genome. More particularly, the regions of homology comprise the recombination sequence motif of the present invention. The recombination sequence motif comprises a 5 base pair nucleic acid sequence or multiple repeats thereof (whether in tandem or interspersed with other nucleotides) that increase the frequency of integration of a transgene. The recombination sequence motif generally comprises the sequence 5′-TATTA-3′, its complement 3′-TAATA -5′, or imperfect variations of such motif differing by a nucleotide, e.g. 5′-GATTA-3′, or a plurality of such motifs, more particularly (TATTA)ₙ or (TAATA)ₙ, where n is between about 2 and about 40, preferably between about 4 and about 20 and more preferably between about 6 and about 10. In an alternate embodiment, the plurality of recombination sequence motifs may be interspersed with other nucleotides in the manner of X-(TATTA)ₚ-X-(TATTA)ₚ-X, where X is any sequence of nucleotides between about 1 and about 10 nucleotides in length and p is between about 1 and about 20. In a further embodiment of the invention, the recombination sequence motif comprises at least one segment of AᵣTᵣ rich repeated sequences where r is between about 5 and about 50, more preferably between about 10 and about 25 and the AT rich segment is between about 20 and about 200 base pairs in total length more preferably between about 50 and about 100 base pairs in total length.

As previously stated, plastid vectors are designed to target transgene integration into the plastid genome via homologous recombination. The location of transgene insertion must be chosen such that the insertion does not cause any disruption of normal plastid function. This is achieved by cloning the transgenes into a plastid intergenic region where no unidentified open reading frames exist, preferably such that readthrough transcription from the transgenes into neighboring resident plastid genes is avoided. Two plastid genomic locations are targeted for insertion of transgenes: the Large Single Copy region and the Inverted Repeat region. Because the Inverted Repeat region is present in two copies per genome, the transgenes will also be present in two copies in transformed lines.

a) Large Single Copy Region

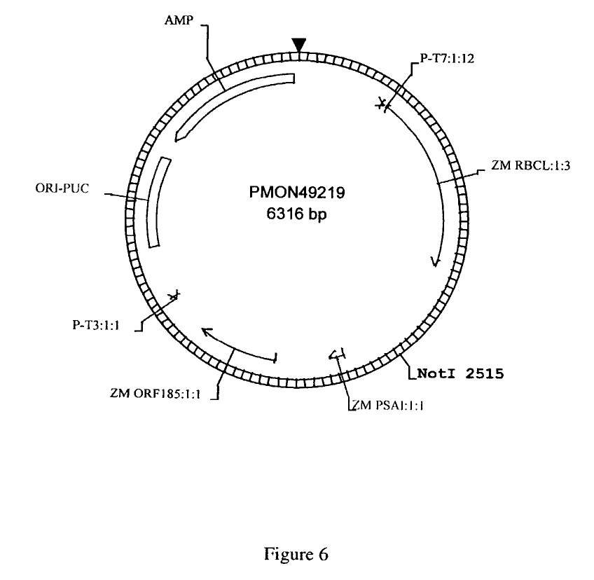

In tobacco, the site between the rbcL and accD genes in the tobacco Large Single Copy region was shown to be a successful insertion site (Svab and Maliga, 1992). Based on success in tobacco, the maize plastid genomic region downstream of rbcL is suitable as a site of insertion for maize plastid transgenes. However, the gene order between tobacco and maize in this genomic region is different. Complete nucleotide sequencing of the maize plastid genome (Maier et al., 1995 J. Mol. Biol. 251:614-628; Genbank accession X86563) revealed that a large inversion occurred during evolutionary separation of the monocot and dicot plastid lineages such that the region downstream of rbcL is completely different between tobacco and maize. Therefore, insertion of transgenes in this region requires thoughtful identification of a suitable non-coding, intergenic region. And an ˜3.4 kb region surrounding the maize plastid rbcL gene was cloned for use as a homologous targeting sequence to direct transgene insertion. To facilitate cloning of transgenes into the homologous flanking region, a unique NotI restriction site was engineered ˜580 bp downstream of rbcL in the rbcL-psaI intergenic region, away from any small open reading frames. The NotI insertion site is flanked by ˜1.5 kb and ˜-1.8 kb of homologous DNA on either side of the transgenes to direct integration into the plastid genome by homologous recombination. Plasmid pMON49219 shown in FIG. 6 carries this ˜3.4 kb plastid fragment with the engineered NotI site.

b) Inverted Repeat Region

A second targeting location for insertion of maize transgenes is upstream of trnV, in the intergenic region between the trnV/rrn16 operon and the divergently transcribed rps12/3′-rps7 operon. This region is located in the Large Inverted Repeat region and is therefore present in two copies per plastid genome. The analogous region is routinely used for transgene insertion in tobacco (Staub and Maliga, 1993; Zoubenko et al., 1994 Nucleic Acids Res. 22:3819-3824). However, the sequence and gene content at the site of insertion differs between tobacco and maize.

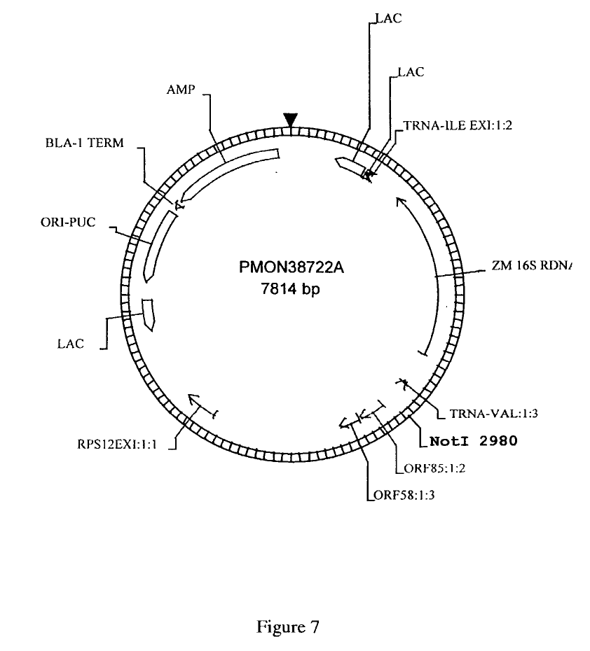

An ˜4.8 kb plastid DNA fragment was chosen for PCR amplification and cloning as the maize homologous flanking region. As a result of PCR amplifications, a unique NotI insertion site was created approximately mid-way between the ˜4.8 kb homologous flanking region, with ˜2.3 kb and ˜2.5 kb of homologous DNA on either side of the transgene insertion site. The pMON38722 clone with this maize flanking region and unique NotI site is shown in FIG. 7.

In tobacco, the transgene insertion site in the analogous region of the plastid genome disrupts an open reading frame of no known function that is not found in other plant species. Insertion into this location in tobacco has no deleterious effect (Staub and Maliga, 1992). In maize, two different unidentified open reading frames, each with no known function, are present in the analogous position. Therefore, it is unknown whether insertion into these open reading frames would affect plastid gene functions. To avoid any possible effect on plastid gene function, the insertion site was chosen in the intergenic region between the trnV gene and the nearby ORF85 gene, away from any putative plastid gene regulatory elements.

For selection of plastid transformed cells, the aadA gene that gives resistance to spectinomycin and streptomycin or a gene that confers tolerance to glyphosate was chosen.

For plastid transformation vectors carrying aadA, streptomycin was used as the selective agent because maize plastids are naturally resistant to spectinomycin. Selection using this antibiotic is based on inhibition of plastid protein synthesis, which prevents accumulation of photosynthetic proteins and chlorophyll, thus resulting in bleaching. Resistant cells are identified by their green color on selective media. Therefore, this antibiotic can only be used with light-grown, chlorophyll-containing green tissue culture systems.

Glyphosate is also used as a selective agent. Glyphosate inhibits aromatic amino acid biosynthesis but also has more pleiotropic effects including bleaching and inhibition of growth. Resistant cells are differentiated by growth and by greening if grown in the light. Therefore, this selectable marker is useful for both dark-grown or light-grown tissue culture systems.

For expression of the aadA or transgenes capable of conferring glyphosate tolerance, expression signals that provide for constitutive transcription and translation of the transgene are most desirable. The expression signals need to be derived from resident maize plastid genes to ensure that the appropriate trans-acting factors are present for faithful gene expression. In most cases, we have used the maize 16S ribosomal RNA (rrn16) operon promoter (ZmPrrn) to drive transgene expression. This promoter region includes the mapped transcriptional start site of the rrn16 operon (Strittmatter et al., 1985 EMBO J, 4 (3): 599-604).

To facilitate identification of plastid transformants, a GFP transgene may be included in the transformation vectors. GFP can be monitored in live tissue and used to follow the growth of transplastomic sectors (Sidorov et al. 1999 Plant Journal 19:209-216). The GFP transgene may also be driven by the ZmPrrn promoter. The translational enhancer sequence, G10L (Staub et al. 2000) may be included to ensure constitutive high level translation.

Additional expression cassettes can comprise any nucleic acid to be introduced into a host cell plastid by the methods encompassed by the present invention including, for example, DNA sequences or genes from another species, or even genes or sequences that originate with or are present in the same species, but are incorporated into recipient cells by genetic engineering methods rather than classical reproduction or breeding techniques. An introduced piece of DNA can be referred to as exogenous DNA. Exogenous as used herein is intended to refer to any gene or DNA segment that is introduced into a recipient cell, regardless of whether a similar gene may already be present in such a cell. The type of DNA included in the exogenous DNA can include DNA that is already present in the plant cell, DNA from another plant, DNA from a different organism, or a DNA generated externally, such as a DNA sequence containing an antisense message of a gene, or a DNA sequence encoding a synthetic or modified version of a gene.

Plant plastids are targeted for transformation by particle bombardment methods. The tissue to be bombarded preferably contains a sufficient number of plastids to effect transformation and from which plants may be regenerated. The tissue may include leaves, stem, roots, callus, embryos, or other tissue types. In one embodiment, actively proliferating meristematic areas from the green callus is selected, and leaves or leaf primordia are removed from one to a few days prior to particle gun bombardment. The selected calli are then transferred into the middle of the plates with solid MS2 (MS medium (Murashige and Skoog, 1962 Physiol. Plant 15:473-479) supplemented with 40g/l maltose, 500 mg/l casein hydrolysate, 1.95 g/l MES, 2 mgA BA, 0.5 mg/l 2,4-D, 100 mg/l ascorbic acid, pH 5.8) or MS3 (same as MS2, except that BA is 1 mg/l and 2,4-D is replaced by 2.2 mg/l picloram) medium. The prepared plates were incubated at light conditions.

The plasmid DNA is precipitated with gold (0.4 or 0.6 μm) or tungsten particles according to standard operation procedure for a helium gun. Particles (0.5 mg) are sterilized with 100% ethanol, washed 2-3 times each with 1 mL aliquots of sterile water. They are then resuspended in 500 μL of sterile 50% glycerol. Twenty-five μL aliquots of particle suspension are added into 1.5 mL sterile microfuge tubes, followed by 5 μL of DNA of interest (at 1 mg/mL) and mixing by finger-vortexing. A fresh CaCl₂ and spermidine premix is then prepared by mixing 2.5M CaCl₂ and 0.1M spermidine at a ratio of 5 to 2. Thirty-five μL of the freshly prepared “premix” is added to the particle-DNA mixture, and mixed quickly by finger-vortexing. The mixture is incubated at room temperature for 20 min. The supernatant is removed after a pulse spin in the microfuge. The DNA-particles mixture is washed twice, first by resuspending in 200 μL of 70% ethanol, followed by pulse spin and removing of the supernatant, repeated with 200 μL of 100% ethanol. The DNA-particles mixture is finally resuspended in 40 μL of 100% ethanol. After thorough mixing, an aliquot of 5 μL is loaded onto the center of the macrocarrier already installed in the macrocarrier holder, and allowed to dry in a low humidity environment, preferably with desiccant. Each tube can be used for 5-6 bombardments.

In comparison to the standard protocol, double the concentration of DNA was also used for particle preparation. The plate with the target tissue is bombarded twice using the helium gun (Bio Rad, Richmond, Calif.) using the protocol described by the manufacturer, at target shelf levels L3. The gap distance is set at 1.0 cm and the rupture pressures at 1100-1550 psi.

To analyze the resulting transformed tissue, a small portion of the transformed sector is sacrificed for DNA extraction and used for PCR analysis. The sample is ground in 150 μL of CTAB and incubated at 65° C. for 30 minutes. The mixture is then cooled to room temperature and extracted with 150 μL of chloroform:isoamyl alcohol (24:1). The mixture is spun at 14,000 rpm for 10 minutes. The supernatant is collected to the new tube and two volumes of 100% ethanol are added. The solution is then kept at −20° C. for 30 minutes to precipitate the nucleic acids. DNA is spun down at 14,000 rpm for 10 minutes. The DNA pellet is then washed with 75% ethanol, air dried, and dissolved in 24 μL of water.

PCR reactions are performed using Roche Expand Long Template PCR System. Two microliters of the above DNA solution is used as the template. The other components are used according to the manufacturer's recommendations. The PCR mixture is first denatured at 94° C. for 2 minutes, then repeated 35 cycles of 94° C. for 10 sec, 53° C. for 30 sec and 68° C. for 2 minutes, and at last elongated at 68° C. for 10 minutes. The PCR products are then separated on 1% agarose gel.

Putatively transformed sectors are normally kept on selection to allow them to continue growth so that they can be regenerated. Under such circumstances, a couple of approaches will be taken to rescue the putative sectors. First, because gfp gene is included in most of the vectors, GFP can be used as a “screenable” marker. The GFP positive sectors can be isolated under a dissecting microscope and placed on medium without any selective agent to allow the sectors to recover and to continue to grow by monitoring GFP expression; or the dissected sectors can be placed on media alternating with and without the selection to allow continue growth of the sectors without running the risk of losing the transformed copies of the plastid genomes; or the dissected sectors could be put on medium with low levels of selection to balance the growth and maintenance of the transformed copies. Second, the dissected sectors could also be placed on top of nurse cultures, which are resistant to glyphosate, in the presence of glyphosate for a period of time to help the putative sectors to grow and to amplify its transgenic copies of the plastid genome.

The invention now being generally described, it will be more readily understood by reference to the following examples that are included for purposes of illustration only and are not intended to limit the present invention.

### EXAMPLES

### Example 1

A plastid transformation vector containing a transgene is prepared comprising homologous flanking sequences capable of causing the integration of the transgene into the plastid genome wherein the homologous flanking sequence include sequences with naturally occurring repeats of the recombination sequence motif TATTA. For a tobacco plastid vector, sequences would be used that have homology to the Prrn promoter, specifically including sequences at position 101756-101820 or 140821-140875 in the inverted repeat (numbers correspond to the sequence of the tobacco chloroplast genome, Genbank accession Z00044). The transgene in the vector is then capable of being targeted to the TATTA repeats in the tobacco chloroplast genome upon transformation of the vector into the tobacco chloroplast.

### Example 2

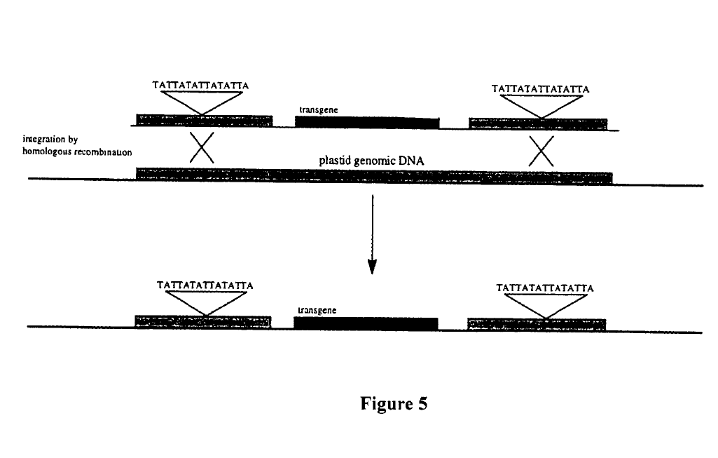

Engineered TATTA repeats may be used as integration target sites rather than naturally occurring TATTA sequences. Transplastomic lines are made using state of the art plastid transformation technologies. The transgenic DNA that is inserted into the plastid genome is designed to include repeats of the TATTA sequence at one or both ends of the transgene insertion as shown in FIG. 5. Homoplasmic plants made with such a primary transgene construct could then been used as an explant source for secondary transformations, in which the transgenes to be integrated would be flanked by sequences homologous to the fragments in which the TATTA sequences reside. The secondary construct may also have the TATTA repeats, in which case the final product will contain TATTA repeats, or it will lack the TATTA repeats (but otherwise have perfect homology to the flanking sequences) in which case the final product may lack the TATTA repeats. The secondary construct will preferably have a different selectable marker than the other. Alternatively it may be possible to reuse the first selectable marker if, for example, a site-specific recombination system (e.g. Cre/lox) is used to remove the first selectable marker gene, in which case the first transgene construct would have to be engineered to include properly configured site-specific recombination sites.

### Example 3

This example describes how the recombination sequence motif of the present invention was identified. A plastid transformation vector was constructed, pMON53119, which is a derivative of pMON30125 (Sidorov et al., 1999) which is based on pPRV100B (Zoubenko et al., 1994). It contains the GFP coding region (Pang et al., 1996) driven by the Prrn promoter (Svab and Maliga, 1993) and the Trps16 (Staub and Maliga, 1994) 3′-end required for mRNA stability. The aadA gene flanked by lox sites in direct repeat orientation is driven by the PpsbA and TpsbA expression elements (Staub and Maliga, 1994). The lox sites were generated by annealing complementary oligonucleotide pairs to create linkers. The sequences of

| | | |
|---|---|---|
| pair #1, | 5′-AGCTTCCATGGATAACTTCGTATAGCATACATTATACGAAGTTATA-3′, | (SEQ ID NO:1) |
| | 5′-AGCTTATAACTTCGTATAATGTATGCTATACGAAGTTATCCATGGA-3′ and | (SEQ ID NO:2) |
| pair #2, | 5′-AATTGATAACTTCGTATAGCATACATTATACGAAGTTATCCCCATGGC-3′, | (SEQ ID NO:3) |
| | 5′-AATTGCCATGGGGATAACTTCGTATAATGTATGCTATACGAAGTTATC-3′ | (SEQ ID NO:4) |

(loxP sequences underlined). The linkers carrying the loxP sites were cloned adjacent to the aadA gene cassette. An internal NcoI site (bold) within each linker was used to insert the aadA gene cassette into an NcoI site between the Prrn promoter and GFP coding region. The aadA gene cassette is cloned in divergent orientation relative to the GFP gene.

Construction of ctp-cre plant nuclear expression and transformation vectors. A chimeric gene (ctp-cre) encoding the chloroplast transit peptide (CTP) of the Arabidopsis thaliana 5-enolpyruvylshikimate-3-phosphate (EPSP) synthase gene (Klee et al., 1987) fused in frame to the N-terminus of the Cre recombinase from bacteriophage P1 was created by overlap PCR as follows: PCR primers 1615 (5′-GGCCTCTAGAGGATCCAGGAG-3′) (SEQ ID NO:5)and 1870 (5′-ATTGGACATGCACGCCGTGGAAACAGAAGAC-3′) (SEQ ID NO:6)were used to amplify the EPSP synthase CTP, and primers 842 (5-CACGGCGTGCATGTTCAATTTACTGACCGT-3′) (SEQ ID NO:7)and 1208 (5′-TTCGGATCCGCCGCATAACCA-3′) (SEQ ID NO:8) were used to amplify a fragment of the cre gene. The resultant PCR products have 19 bp of overlap between the 3′-end of the EPSP synthase CTP and the 5′-end of the cre gene. The PCR products were subsequently denatured and used as a template in a second PCR reaction with primers 1615 and 1208. In this reaction, the overlap between the CTP and the Cre DNA fragments primed the synthesis of a full length ctp-cre chimeric gene, which is then further amplified by the 1615 and 1208 primers for use in vector construction. The PCR construction was confirmed by sequence analysis.

The ctp-cre coding region was cloned into plant expression vectors, and subsequently into an Agrobacterium binary plant transformation vector. The binary vector is based on pMON 18462 (Pang et al., 1996), and carries the nptII selectable marker gene driven by the nopaline synthase (nos) promoter, with the nos 3′ terminator. The ctp-cre gene in transformation vector pMON49602 is driven by the e35S promoter (Kay et al., 1987), whereas the Arabidopsis thaliana ACT2 gene promoter including the intron in the 5′ untranslated region (An et al., 1996) is used in vector pMON53147. Both ctp-cre genes utilize the nos 3′ terminator. The binary vectors were assembled in Escherichia coli and transferred into Agrobacterium tumefaciens strain ABI by electroporation.

Growth of Plants and Selection for Transformants

Nicotiana tabacum cv. Petit Havana (tobacco) was maintained aseptically on phytagel-solidified medium containing MS salts (Murashige and Skoog, 1962), Gamborg's B5 vitamins (Gamborg et al., 1968) with 3% sucrose at 24° C. with 16 hr photoperiod. Plastid transformation, selection and regeneration of plants transformed with vector pMON53119 was as described (Svab and Maliga, 1993). Several independent transformants were carried to homoplasmy as judged by Southern blot analysis. A single homoplasmic line was chosen for nuclear retransformation by Agrobacterium. The parental plastid transformed Nt-53119 line was used for nuclear retransformation via the Agrobacterium-mediated leaf-disk transformation protocol (Horsch et al., 1985) using vectors pMON49602 (carrying e35S:ctp-cre) and pMON53147 (carrying Act2:ctp-cre). Independent nuclear retransformants were selected on kanamycin sulfate (50 ug/mL). Retransformed shoots were considered primary transformants. Plants regenerated from leaves of the primary shoots were termed subclones.

Fluorescence Microscopy

Kanamycin resistant shoots from Agrobacterium-mediated transformation were monitored for activation of the GFP reporter gene by visual detection of fluorescence using a Leica MZ-8 microscope with GFP Plus Fluorescence module no. 10446143-143. Shoots showing GFP fluorescence were dissected and transferred onto fresh medium containing kanamycin (50 ug/mL). Individual GFP positive shoots were considered independent primary retransformants.

Gel Blot Analysis

Total cellular DNA isolation and Southern blot analysis was performed according to Sidorov et al (Sidorov et al., 1999). DNA for Southern blot analysis was digested with BamHI restriction endonuclease. Total cellular RNA was isolated and Northern blot analysis performed according to Hajdukiewicz et al. (Hajdukiewicz et al., 1997). PCR amplification and cloning of recombination events

PCR amplification of recombination events was performed using total cellular DNA from nuclear retransformed transplastomic tobacco plants. The primers used for PCR were 5′-GGGCATGCCGCCAGCGTTCATCCTGAGCCAGG-3′ (SEQ ID NO:9) and 5′-GGGGATCCCAAATTGACGGGTTAGTGTGAGCTTATCC-3′ (SEQ ID NO: 10), starting at positions 139,818 and 141,091, respectively, in the tobacco plastid genome [(Shinozaki and Sugiura, 1986); GenBank accession Z00044]. PCR was performed using the Hybaid Omn-E machine. Samples were denatured, annealed and extended at 94° C., 58° C., and 68° C. for 15″, 30″ and 90″, respectively, for 35 cycles. PCR products were digested with BamHI and SphI restriction endonucleases, gel purified and ligated into a pUC vector. The entire nucleotide sequence of the PCR product was determined by dideoxy sequencing (dye terminator kit, Perkin Elmer).

Segregation Analysis

Retransformants were grown to maturity in the greenhouse and allowed to set self seed. Seed pods were surface sterilized with 100% ethanol and dried. The seeds were plated onto medium containing either kanamycin monosulfate (200 ug/mL) or spectinomycin dihydrochloride (500 ug/mL). After 2-3 weeks seedling phenotype was scored as resistant (green) or sensitive (bleached) to the antibiotics.

In addition to the expected recombination between loxP sequences, Cre activity apparently also stimulated a general recombination pathway in plastids that revealed a “hotspot” for recombination. While analyzing Cre-mediated deletion events, a class of deletions that represented recombination between the introduced loxP site and endogenous plastid genome sequences. Southern blot analysis showed that the recombination events were focused at a discrete position in the plastid genome, ˜500 base pairs away from the loxP site, indicating the presence of a hotspot. Three products of this class of recombinants were cloned and sequenced. All three were found to occur within a region of the plastid genome that contains numerous direct repeats of the recombination sequence motif 5′-TATTA-3′, located downstream of the transcriptional start site of the rps7/3′-rps12 operon promoter in tobacco chloroplasts (positions 101756-101820 or 140821-140875 in the Inverted Repeat of the tobacco chloroplast genome, Genbank accession Z00044). The hot-spot region consists of numerous copies of a small directly repeated motif, TATTA. It should be noted that the hot-spot sequences do not appear to function as “cryptic” lox sites, that have been reported in some much larger genomes (Sauer, 1992; Thyagarajan et al., 2000), because testing of the pMON53119 plasmid in E. coli that overexpress Cre showed exclusively the correct 4.3 kb excision event (data not shown). Furthermore, the sequences have very little homology with loxP, particularly in the spacer region shown to be critical for normal Crellox recombination (Hoess et al., 1986; Lee and Saito, 1998).

### Example 4

The recombination events that were recovered in Example 3 involving the hotspot sequences were stimulated by Cre. They did not occur in the absence of Cre, and the junction involved one of the two loxP sites. It is possible, therefore, that Cre recombinase acts on the hotspot sequence. Thus, it should be possible to stimulate integration of transgenes at the hotspots sequences by expressing Cre recombinase during the transformation process. Cre recombinase can be expressed by a plastid functional promoter. Such promoters include, but are not limited to, the promoter of the D1 thylakoid membrane protein, psbA (Staub et al. EMBO Journal, 12(2):601-606, 1993), and the 16S rRNA promoter region, Prrn (Svab and Maliga,1993, Proc. Natl. Acad. Sci. USA 90:913-917). The expression cassette(s) can include additional elements for expression of the protein, such as transcriptional and translational enhancers, ribosome binding sites, and the like. In this example, The Cre expression cassette would be on a separate DNA molecule, or contained within the DNA molecule that also contain additional nucleic acid sequences comprising regions of homology for integration into the host plant cell plastid. More particularly, the regions of homology comprise the recombination sequence motif of the present invention. The recombination sequence motif comprises a 5 base pair nucleic acid sequence or multiple direct repeats thereof that increase the frequency of integration of a transgene. In this example, Cre protein will contact the hotspot sequences, stimulating recombination with regions of homology on the extrachromosomal DNA to be inserted, resulting in higher efficiencies of integration.

What is claimed is:

1. A nucleic acid recombination sequence motif selected from the group consisting of 5′-TATTA-3′ and 3′-TAATA-5′ and single nucleotide variations of said motifs.

2. A nucleic acid sequence comprising a plurality of nucleic acid recombination sequence motifs selected from the group consisting of (TATTA)ₙ and (TAATA)ₙ, where n is between 2 and 40.

3. The nucleic acid sequence of claim 2 wherein the recombination sequence motifs are joined in tandem repeats.

4. The nucleic acid sequence of claim 2 wherein the recombination sequence motifs are interspersed with other nucleotides according to the formula X-(TATTA)ₚ-X-(TATTA)ₚ-X, where X is any sequence of nucleotides between 1 and about 10 nucleotides in length and p is between 1 and about 20.

5. A nucleic acid recombination sequence motif comprising an AᵣTᵣ repeated sequence where r is between about 5 and about 50 and said AᵣTᵣ repeated sequence is between about 20 and about 100 base pairs in length.

6. A nucleic acid sequence for plastid transformation comprising a plastid functional promoter operably linked to a transgene which is operably linked to a transcript termination region forming an expression cassette, said expression cassette flanked by a plastid region of homology comprising a nucleic acid recombination sequence motifs selected from the group consisting of (TATTA)ₙ and (TAATA)ₙ, where n is between 2 and 40.

7. The nucleic acid sequence of claim 6 wherein said recombination sequence motifs are joined in tandem repeats.

8. The nucleic acid sequence of claim 6 wherein said recombination sequence motifs are interspersed with other nucleotides according to the formula X-(TATTA)ₚ-X-(TATTA)ₚ-X, where X is any sequence of nucleotides between 1 and about 10 nucleotides in length and p is between 1 and about 20.

9. A transplastomic plant comprising a recombination sequence motif integrated into its plastid genome for use as a transformation integration site, said recombination sequence motif selected from the group consisting of (TATTA)ₙ and (TAATA)ₙ, where n is between 2 and 40.

10. The plant of claim 9 wherein said recombination sequence motifs are joined in tandem repeats.

11. The plant of claim 9 wherein said recombination sequence motifs are interspersed with other nucleotides according to the formula X-(TATTA)ₚ-X-(TATTA)ₚ-X, where X is any sequence of nucleotides between 1 and about 10 nucleotides in length and p is between 1 and about 20.


SEQUENCE LISTING (10 sequences)

SEQ ID NO: 1 - DNA, 46 bp, Artificial Sequence
  synthetic construct
agcttccatggataacttcgtatagcatacattatacgaagttata

SEQ ID NO: 2 - DNA, 46 bp, Artificial Sequence
  synthetic construct
agcttataacttcgtataatgtatgctatacgaagttatccatgga

SEQ ID NO: 3 - DNA, 48 bp, Artificial Sequence
  synthetic construct
aattgataacttcgtatagcatacattatacgaagttatccccatggc

SEQ ID NO: 4 - DNA, 48 bp, Artificial Sequence
  synthetic construct
aattgccatggggataacttcgtataatgtatgctatacgaagttatc

SEQ ID NO: 5 - DNA, 21 bp, Artificial Sequence
  synthetic construct
ggcctctagaggatccaggag

SEQ ID NO: 6 - DNA, 31 bp, Artificial Sequence
  synthetic construct
attggacatgcacgccgtggaaacagaagac

SEQ ID NO: 7 - DNA, 30 bp, Artificial Sequence
  synthetic construct
cacggcgtgcatgttcaatttactgaccgt

SEQ ID NO: 8 - DNA, 21 bp, Artificial Sequence
  synthetic construct
ttcggatccgccgcataacca

SEQ ID NO: 9 - DNA, 32 bp, Artificial Sequence
  synthetic construct
gggcatgccgccagcgttcatcctgagccagg

SEQ ID NO: 10 - DNA, 37 bp, Artificial Sequence
  synthetic construct
ggggatcccaaattgacgggttagtgtgagcttatcc